(19) United States
(12) Patent Application Publication
(10) Pub. No.: US 2026/0283192 A1
(43) Pub. Date: Sep. 24, 2026

(54) IMPROVED PEA OR FABA BEAN PROTEINS

(71) Applicant: ROQUETTE FRERES, Lestrem (FR)

(72) Inventor: Guillaume FARDIN, Lestrem (FR)

(21) Appl. No.: 19/155,055

(22) PCT Filed: Feb. 9, 2024
(86) PCT No.: PCT/EP2024/025073

§ 371 Date: Aug. 8, 2025

(30) Foreign Application Priority Data
Feb. 10, 2023 (FR) 2301279

(51) Int. Cl. A23J 1/14; A23L 5/20; C12N 1/205; C12N 9/24; C12R 1/125

(52) U.S. Cl. CPC A23J 1/148; A23L 5/25; C12N 1/205; C12N 9/2402; C12R 2001/125; C12Y 302/01022; C12Y 302/01026

(57) ABSTRACT

The present invention relates to pea proteins having a milky aromatic profile, to a method for producing said pea proteins, and to the use of said proteins for producing food or beverage products, in particular plant-based alternatives to milk.

### FIELD OF THE INVENTION

The invention relates to a new composition comprising pea or faba bean proteins associated with soluble fibers. Another object of the invention relates to a method for producing these new compositions of pea proteins. The invention also relates to the use of said compositions of pea or faba bean proteins in the production of food products.

### PRIOR ART

Daily requirements for proteins are generally between 12 and 20% of food intake. These proteins are provided equally by products of animal origin (meat, fish, eggs, dairy products) and by plant-based food (cereals, leguminous plants, seaweed).

In developed countries, protein intake is still today predominantly in the form of proteins of animal origin. These proteins have good nutritional properties and interesting functional properties, which allow them to be used in a very wide variety of food products.

However, numerous studies show that excessive consumption of proteins of animal origin to the detriment of plant proteins is one of the causes of increases in cancer and cardiovascular diseases. Moreover, animal proteins have many drawbacks, both in terms of their allergenicity (especially proteins from milk or eggs) and in environmental terms, in connection with the harmful effects of intensive farming.

Thus, there is an increasing demand from manufacturers for proteins of plant origin having beneficial nutritional and functional properties without, however, having the disadvantages of proteins of animal origin.

Since the 1970s, the pea is the pulse plant which has been the most developed in Europe, predominantly in France, especially as a protein resource intended for animal and human food. The pea contains approximately 27% by weight of protein substances. The term “pea” is considered here in its broadest accepted use and includes, in particular, all the wild varieties of “smooth pea” and all the mutant varieties of “smooth pea” and “wrinkled pea”, regardless of the uses for which said varieties are usually intended (human food, animal feed and/or other uses). Pea protein, predominantly pea globulin, has been extracted and utilized industrially for a great number of years.

The field bean, or faba bean, is also a well-known plant of the Vicia faba species. It is a leguminous plant of the Fabaceae family, Faboideae subfamily, Fabeae tribe. This is the same species as the broad bean, a plant that has been used for human consumption since ancient times. The word bean thus refers to both the seed and the plant.

Firstly, there are the so-called “dry extraction” methods for pea or faba bean protein. The principle of these methods is to mill the seed into flour, which is then introduced into a turbo separator, a device used to classify particles according to their size and density within an air stream. Turbo separation produces a protein-enriched fraction and a starch-enriched fraction. As will be explained later, the protein-enriched fraction is about 40%-60% and still contains between 2% and 15% starch. This starch is a polysaccharide fraction that is not necessarily desired as it contributes to raising blood sugar levels. Its replacement by polysaccharides that are non-digestible by the consumer but digestible by their digestive microflora would be of interest. These same properties are also observed in the case of the faba bean.

Another example of a pea or faba bean protein extraction method is patent EP1400537 which is typical of so-called “wet” extraction methods. In this method, the seed is milled in the absence of water (method referred to as “dry milling”) in order to obtain a flour. This flour is then suspended in water at room temperature to then proceed with the various steps for extracting the protein. This type of method separates proteins belonging to the globulin subgroup (about 80% of pea proteins) and proteins belonging to the albumin subgroup (about 20% of pea proteins) by isoelectric precipitation. These remain in the liquid fraction after recovery of the floc, which is mainly composed of globulins. These albumins are in solution with galactooligosaccharides (GOS) from the pea which are not easily digested by humans, salts, mainly potassium, which can be harmful in high doses, and other anti-nutritional factors such as anti-trypsin factors. These same properties are also observed in the case of the faba bean.

Several problems are easy to guess such as for example proteins being separated into two fractions requiring two different fractions to be valorized, or else the GOS being combined with albumins which are difficult to digest and require post-treatment which makes the method more complex and costly. Lastly, the massive presence of starch in the protein-enriched fraction of concentrates obtained by dry turbo-separation is also a nutritional disadvantage for certain formulations.

After a great deal of research, the Applicant has thus developed a new production method that makes it possible to provide a composition which contains all of the so-called soluble proteins of the pea or the faba bean, that is comprising all of the globulins and albumins, as well as an amount of soluble fibers derived from GOS making it possible to obtain a high-quality nutritional source.

### SUMMARY OF THE INVENTION

Thus, the invention relates to a method for producing pea or faba bean protein comprising the following steps:
1. Preparing an aqueous suspension of ground pea or faba bean seeds in an aqueous solution, said preparation being optionally performed in the presence of a heat treatment;
2. Removing an insoluble fraction by solid/liquid separation of the aqueous suspension of ground pea or faba bean seeds obtained in step 1) to obtain a protein-enriched fraction;
3. Defructosylating galactooligosaccharides from the protein-enriched fraction by enzymatic and/or fermentative means.

Preferably, the aqueous suspension of ground pea or faba bean seeds is obtained by adding a pea or faba bean flour obtained by dry milling prior to its dispersion in the aqueous solution.

When the pea or faba bean seeds are introduced as whole pea or faba bean seeds into the aqueous solution, step 1 of the method comprises a step of wet milling the aqueous composition comprising the pea or faba bean seeds in order to obtain the aqueous suspension of ground pea or faba bean seeds.

In the absence of the optional heat treatment in step 1, the aqueous solution and the resulting aqueous suspension are at a temperature between 5° C. and 30° C., preferably at ambient temperature. Ambient temperature is understood to mean a temperature between 15° C. and 27° C., preferentially between 19° C. and 23° C., where the temperature values can be 15° C., 16° C., 17° C., 18° C., 19° C., 20° C., 21° C., 22° C., 23° C., 24° C., 25° C., 26° C. or 27° C.

Preferably, the optional heat treatment of step 1 comprises:
a) introducing pea or faba bean seeds or ground pea or faba bean seeds into an aqueous solution at a temperature of between 65° C. and 90° C. in order to obtain an aqueous composition comprising ground pea or faba bean seeds;
b) heat treating the aqueous composition obtained in step a) at a temperature of between 40° C. and 65° C. for 1 to 10 min.

Preferably, the defructosylation of galactooligosaccharide (GOS) in step 3 is carried out with an enzyme selected from invertase, alpha-galactosidae, beta-fructosidase and any combination thereof.

Alternatively, the defructosylation of galactooligosaccharide (GOS) is carried out using a microorganism of the Bacillus genus, preferentially Bacillus subtilis, preferentially a Bacillus subtilis strain as deposited with the CNCM on May 28, 2020 under number 1-5515.

Another object of the invention relates to the pea or faba bean protein characterized in that it comprises a protein content of between 50% and 70%, expressed in grams of protein per 100 g of dry matter, said proteins consisting of a mixture of globulins and albumins, and between 3% and 15% of soluble fibers derived from pea galactooligosaccharides.

Preferably, the dry weight ratio of globulins/albumins is between 70:30 and 90:10, preferentially between 75:25 and 85:15.

Preferably, the soluble fibers derived from galactooligosaccharides of the pea or faba bean are selected from the list containing melibiose, manninotriose, verbascotetraose and mixtures thereof.

Another object of the invention also relates to the use of said composition of pea or faba bean protein according to the invention for producing food or beverage products, especially plant-based alternatives to milk.

### DETAILED DESCRIPTION OF THE INVENTION

The invention relates to a method for producing a pea or faba bean protein comprising the following steps:
1. Preparing an aqueous suspension of ground pea or faba bean seeds in an aqueous solution, said preparation being optionally performed in the presence of a heat treatment;
2. Removing an insoluble fraction by solid/liquid separation of the aqueous suspension of ground pea or faba bean seeds in step 1) to obtain a protein fraction;
3. Defructosylating galactooligosaccharides from the protein fraction by enzymatic and/or fermentative means.

Step 1) comprises introducing peas or faba beans into an aqueous solution. The pea or faba bean seeds used in step 1) may have been previously subjected to steps that are well known to the skilled person, such as especially cleaning (removal of undesired particles such as stones, dead insects, soil residues, etc.) or even the removal of the external fibers of the pea or faba bean (external cellulose hull) through a well-known step referred to as “dehulling”.

Thus, the term “pea” or “faba bean” in step 1) is understood to mean whole peas or faba beans or in the form of cotyledons, from which the external hull has preferentially been removed. Alternatively, ground pea seeds (that is pea flour) can be used, these ground peas generally being obtained by dry milling.

The aqueous solution may be water and may optionally comprise additives such as especially anti-foaming compounds, salts or bacteriostatic compounds.

The ratio by weight of amount of peas to amount of aqueous solution in step 1) can especially be between 0.1 and 2. In the embodiment where the pea or faba bean seed is ground into flour before being introduced into the aqueous solution, the ratio by weight of the amount of peas to the amount of aqueous solution in step 1) will be between 0.1 and 0.3, preferentially between 0.15 and 0.25. Expressed as a percentage of dry matter, this ratio will be between 10% and 30%, preferentially between 15% and 25%.

According to one variant, the pH of the suspension of step 1) is adjusted to between 8 and 10. This adjustment can be made by adding a base such as sodium hydroxide, lime or potash, preferentially sodium hydroxide. According to another variant, the pH is not adjusted in this step.

The aqueous suspension of ground pea or faba bean seeds is obtained in step 1) by introducing the pea or faba bean seeds or the ground pea or faba bean seeds into the aqueous solution, which may optionally have been previously heated to a temperature of between 65° C. and 90° C. if heat treatment is used.

In the case of heat treatment, the temperature of the aqueous solution can be adjusted beforehand to a temperature between 65° C. and 90° C. Heating can be carried out using any installation well known to the skilled person such as an immersion heat exchanger. Preferentially, the temperature is between 70° C. and 80° C., or even about 75° C.

Alternatively, the aqueous solution does not undergo any prior heating and is heated directly to a temperature of between 65° C. and 90° C. when mixed with the pea or faba bean seeds or ground pea or faba bean seeds. Preferably, the heat treatment temperature is between 40° C. and 60° C., or even between 45° C. and 55° C. Preferentially, the heat treatment is carried out for 2 to 4 min.

According to either of these embodiments, the method then comprises heat treating the suspension formed between the pea or faba bean seeds or the ground pea or faba bean seeds and the aqueous solution to a temperature of between 40° C. and 65° C. for 1 to 10 minutes. The aqueous composition comprising pea or faba bean seeds or ground pea or faba bean seeds can be heated or cooled to reach this temperature.

In the case where pea or faba bean seeds (and not already ground pea or faba bean flour) are used in the preparation of the suspension according to step 1), the method comprises a step of wet milling the aqueous composition comprising pea or faba bean seeds or ground pea or faba bean seeds in order to obtain an aqueous suspension of ground pea or faba bean seeds. If heat treatment is applied, wet milling takes place after said heat treatment. Preferably, the method is carried out using pea or faba bean seeds and the wet milling step is carried out by continuous passage through one or more mills in order to obtain the aqueous suspension of ground pea or faba bean seeds. The one or more mills can be any type of mill suitable for wet milling, such as wet ball mills, wet conical mills, wet helical mills or wet mills equipped with rotor-stator systems. According to one variant, the mill can be the one used in the examples of document WO2019/053387 in the name of the Applicant. In the variant where the mill is of the rotor-stator type, this type of mill can allow continuous milling by passing the aqueous composition obtained between the pea or faba bean seeds and the aqueous solution through said mill. According to one preferred sub-variant, the method combines two cutting stages (pre-cutting then cutting) using different rotor-stator mills for each one of these cuts. The pre-cutting then the cutting can be carried out one after the other or, alternatively, the cutting can take place after pre-cutting then storing the aqueous composition obtained between the pea or faba bean seeds and the treated aqueous solution. Such mills are disclosed in document WO2019/158589. Optionally, a dilution with water can be carried out during this step or at the end of this step in order to form the aqueous suspension of ground pea or faba bean seeds. According to one variant, during milling, water is added continuously or discontinuously to dilute the aqueous suspension. Generally, the dry matter of the aqueous suspension of ground pea or faba bean seeds ranges from 10 to 30%, for example from 15 to 25%.

Step 2) of the method consists in extracting components from the aqueous suspension of ground pea or faba bean seeds, and in particular in extracting a protein fraction by solid-liquid separation of a so-called insoluble fraction from the aqueous suspension of ground pea or faba bean seeds. According to one variant, before carrying out the solid-liquid separation stage, a stage of adjusting the pH of the aqueous suspension of ground pea or faba bean seeds can be carried out. Thus the solid-liquid separation can take place after adjusting the aqueous suspension of ground pea or faba bean seeds to a pH ranging from 6 to 9, preferably from 8 to 9, most preferentially from 8.5 to 9. This pH adjustment stage can be carried out in a stirred tank. This stage can be shorter or longer, and last from 1 to 240 minutes, for example, generally from 5 to 60 minutes. To perform the pH adjustment, any type of acid and/or base, organic or inorganic, or mixtures thereof, can be added. Examples of acids that can be used include hydrochloric acid, sulfuric acid, citric acid or mixtures thereof. Examples of a base that can be used include sodium hydroxide, potash or lime and mixtures thereof. This addition of base or acid and the pH measurement can be carried out online. The base and/or acid may be in the form of aqueous solutions. Advantageously, prior to this solid-liquid separation, the aqueous suspension of ground pea or faba bean seeds is cooled to a temperature less than 15° C. This temperature can especially range from 4 to 14° C., for example from 10 to 12° C. This cooling step can be carried out using known techniques, such as for example passing the aqueous suspension of ground pea or faba bean seeds through a heat exchanger.

Generally, the protein fraction is the soluble part of the aqueous suspension of ground pea or faba bean seeds and the starch- and fiber-rich fraction is the insoluble part. It is also possible to separate more than two insoluble fractions and, for example, to recover a first insoluble fraction that is richer in starch and a second insoluble fraction that is richer in fiber. Thus, according to one variant of the method, a starch-rich fraction and/or a fiber-rich fraction is recovered from the insoluble part resulting from the solid-liquid separation step 2).

“Starch-rich fraction and fiber-rich fraction” or “insoluble fraction” generally refers to a fraction comprising at least 50% of starch and/or fiber. The methods for quantifying starch and fiber are known to those skilled in the art, and specific methods are indicated later in the description. These fractions are conventionally recovered by the known separation methods. The solid-liquid separation may especially be carried out by means of at least one separation step with a decanter, especially a centrifugal decanter, a centrifuge or else with hydrocyclones. The method can likewise make it possible to recover one or more fiber- and/or starch-enriched fractions that are removed from the suspension and to recover the protein fraction that is useful for the rest of the method of the invention.

This step is key in differentiating concentrates obtained by the dry process. The protein-enriched fraction obtained at the end of this step contains between 0% and 0.5% starch, unlike concentrates obtained by turbo separation which contain between 2% and 15%. This step will have a direct impact on the final composition obtained, as will be discussed later in this application.

At the end of step 2), the method can comprise a step of adjusting the pH of the pea or faba bean protein to a pH ranging from 6 to 7.5, generally from 6.5 to 7.5. This step can be carried out by adding an inorganic or organic base, such as by adding sodium hydroxide. The pH is generally raised by adding a basic aqueous solution.

### Step 3)

The method then comprises a step 3) of defructosylating galactooligosaccharides by enzymatic and/or fermentative means.

For the purposes of this invention, “galactooligosaccharides” refer to oligomers formed from a number n of oses (monosaccharides) by alpha or beta glycosidic bonding and naturally present in the pea or faba bean such as raffinose or stachyose.

Preferably, the water-soluble fraction according to the invention comprises defructosylated galactooligosaccharides selected from the list containing melibiose, manninotriose and verbascotetraose.

The term “melibiose”, for the purposes of the present invention, means the diholoside consisting of a galactose unit bonded to a glucose unit by an α(1→6) osidic bond.

The term “manninotriose”, for the purposes of the present invention, means the triholoside consisting of the chain of one galactose unit bonded by an α(1→6) osidic bond to another galactose unit, itself bonded to a glucose unit by another α(1→6) bond.

The term “verbascotetraose”, also referred to as “manninotetraose”, for the purposes of the present invention, means the tetraholoside consisting of the chain of three galactose units bonded by α(1→6) osidic bonds, the third galactose unit being itself bonded to a glucose unit by another α(1→6) bond.

Any method well known to the skilled person for quantifying these defructosylated galactooligosaccharides is suitable for the purposes of the present invention. Chromatographic methods are preferred. Preferably, a person skilled in the art will use the HPAEC-PAD amperometric dosing method and in particular with the following equipment:
Dionex Carbopac PA1 4*50 mm pre-column—Ref. 43096
Dionex Carbopac PA1 4*250 mm column—Ref. 35391
The detector is of the PAD type, specifically gold cell
The eluents are:
SolventA/NaOH 0.1 M: Stir 4 liters of water under helium (flow rate: 100 mL/mn) for 15 min. Add 20 ml of 50% NaOH. Stir again under helium at 40 ml/min.
Solvent B/NaOH 0.1 M+0.5 M sodium acetate Weigh 82 g of Na acetate directly in the canister. Add 2 l of H2O. Stir under helium (flow rate: 100 ml/min) for 15 min then add 10 ml of 50% NaOH, stir again under helium. The helium flow rate can be lowered to 40 ml/min.

Standards are used to calibrate the HPLC and in particular:

| Reagents | Reference |
|---|---|
| Melibiose | Fluka ref. 63630 |
| Raffinose | Sigma ref. R-0514 |
| Stachyose | Sima ref. S-4001 |
| Verbascose | Fluka ref. 56217 |

An internal standard is also used: Panose ref. SIGMA P-2407 60 mg in 100 ml water.

The injected volume is 5 μl at a temperature of 15° C. The analysis time is 90 min with a column temperature of 30° C. and an injector sensitivity of 300 nC or 5 μA

The chromatographic elution conditions are as follows:

| Time | Flow rate | | |
|---|---|---|---|
| (min) | (ml/min) | Solvent A | Solvent B |
| 0 | 0.5 | 98 | 2 |
| 60 | 0.5 | 95 | 5 |
| 65 | 0.5 | 70 | 30 |
| 65.05 | 0.5 | 0 | 100 |
| 75 | 0.5 | 0 | 100 |
| 75.05 | 0.5 | 98 | 2 |
| 90 | 0.5 | 98 | 2 |

The oxidation program of the PAD detector is as follows:

| Time (min) | Potential | Integration |
|---|---|---|
| 0 | +0.05 | |
| 0.20 | +0.05 | Start |
| 0.40 | +0.05 | End |
| 0.41 | +0.75 | |
| 0.60 | +0.75 | |
| 0.61 | −0.15 | |
| 1.0 | −0.15 | |

The calibration is carried out by preparing curves according to the table below:

| Amount | Melibiose | Raffinose | Stachyose | Verbascose |
|---|---|---|---|---|
| in mg | QSP 50 ml | QSP 25 ml | QSP 25 ml | QSP 25 ml |
| T1 | 10 | 5 | 5 | 5 |
| T2 | 25 | 10 | 10 | 10 |
| T3 | 50 | 15 | 15 | 15 |
| T4 | 75 | 25 | 25 | 25 |
| T5 | 100 | | | |

Take 1 ml of control (from the 2 curves)+1 ml internal standard, q.s. 20 ml of water.

Weigh the amount in mg of sample, add 1 ml of internal standard and adjust to 20 ml of water.

Filter on GxF/GHP 0.45 μm ref. 4559T.

In a first alternative, defructosylation of galactooligosaccharide (GOS) is performed with an enzyme selected from invertase, alpha-galactosidae, beta-fructosidase and any combinations thereof.

Preferably, the enzyme used is an invertase such as for example Sumizyme INV. The pH and the temperature are rectified in order to optimize the defructosylation reaction, such as for example pH 5 and 55° C. for Sumizyme INV.

The reaction is monitored by performing an analysis for example by HPLC analysis of the sugars present in solution. When the defructosylation is sufficient or even complete, neutralize with bases such as sodium hydroxide then perform heat treatment in order to inhibit the enzyme, for example at 130° for 10 seconds.

In a second alternative, the defructosylation of galactooligosaccharide (GOS) is carried out with a microorganism of the Bacillus genus, preferentially Bacillus subtilis, preferentially a Bacillus subtilis strain as deposited with the CNCM on May 28, 2020 under number 1-5515.

For the purposes of the invention, “fermentation” means metabolic processes generally converting carbohydrates into acids, into gases or into alcohols in order to extract some of their chemical energy while re-oxidizing the co-enzymes reduced by these reactions. This is a redox metabolic pathway in which the ultimate electron acceptor is often confused with the final product of the reactions. It is characterized by a partial degradation of the fermentable substance and allows only for a limited production of energy. It takes place in yeasts and bacteria, as well as in muscle cells lacking oxygen, that is under anaerobic conditions.

In this step of the method, it is important to defructosylate GOS without altering the carbon skeleton thereof, so as not to hydrolyze it. There are a number of solutions to solve this technical problem in the background art. The present invention prefers to transform them into fibers beneficial for human and animal nutrition.

Steps 1), 2) and 3) of the method are carried out in this order. However, other optional steps can be implemented between steps 1), 2) and 3), such as pH adjustment, if this is deemed necessary by the skilled person. Preferably, after the defructosylation step, the method may comprise a step 4) of additional heat treatment of the pea or faba bean protein. The temperature and time conditions can vary widely in this step, for example from 70° C. to 140° C. and lasting from 0.1 seconds to several minutes. According to a first variant of this step of additional heat treatment, the temperature ranges from 70° C. to 90° C. and its duration ranges from 0.1 seconds to 30 minutes. According to a second variant of this step of additional heat treatment, the temperature ranges from 90° C. to 110° C. and its duration ranges from 0.1 seconds to 5 minutes. According to another variant, this step of additional heat treatment is carried out at a temperature ranging from 110° C. to 140° C. for a time ranging from 0.1 to 30 seconds, preferentially from 0.2 to 15 seconds, for example from 0.3 to 10 seconds. The aim of this step may be to functionalize and/or sanitize the pea protein. To perform this additional heat treatment step, the pea or faba bean protein can be in the form of an aqueous dispersion, preferentially having a dry matter content ranging from 10 to 25%, for example from 15 to 20%. Advantageously, the method of the invention comprises, following the additional heat treatment step, a step of cooling of the pea protein. According to one preferred variant, this cooling step is obtained by flash-cooling. At the end of this step, the temperature can range from 60° C. to 100° C., for example between 70° C. and 90° C. Similarly, this flash-cooling step is carried out by applying a vacuum to the aqueous dispersion of pea protein, the vacuum applied being determined based on the chosen cooling temperature.

According to one variant of the method, it comprises an optional step 5) of shearing the pea or faba bean protein for example by passing the aqueous dispersion of proteins through a high-pressure pump. As examples of high-pressure pumps, mention may be made of the high-pressure pumps marketed by Silverson, also referred to as “high shear mixers”, for example those in the UHS range. Preferably, the shearing step is performed by a high-pressure pump.

The shearing step can take place before or after the steps of heat treatment and/or raising the pH.

According to another variant, the method alternatively comprises an optional step 5) of homogenizing the pea protein.

To perform this homogenization step, it is possible to use any type of homogenizer. According to the invention, it refers to equipment comprising a high-pressure pump and a homogenizing head, wherein the equipment is designed so that the product to be homogenized passes under pressure through this homogenizing head. A homogenizing head consists of a reduced opening, generally comprising a seat, a valve and an impact ring. Passing the aqueous dispersion of pea or faba bean proteins through the homogenizer can thus make it possible to homogenize the pea protein. The homogenization can be low-pressure homogenization, high-pressure homogenization or ultra-high-pressure homogenization. Depending on the homogenization technique used, the homogenization pressure can vary widely from 1 to 1000 bar, for example from 20 to 800 bar. According to one variant, the homogenization pressure ranges from 20 to 200 bar, for example from 50 to 150 bar. According to another variant, the homogenization pressure ranges from 200 to 800 bar, for example from 300 to 800 bar. According to one variant, the homogenization is a single-action homogenization. According to another variant, the homogenization is a multiple-action homogenization, for example a double-action homogenization. The homogenizers that can be used are marketed by GEA or Tetra Pak, for example.

The homogenization step can take place before or after the steps of heat treatment and/or raising the pH.

The method according to the invention can also comprise an optional step 6) of membrane nanofiltration performed preferably with a cut-off threshold between 150 Da and 300 Da. This step enables the composition to be pre-concentrated prior to any potential subsequent evaporation and/or drying steps, as well as desalting. Tests have shown that with a cut-off threshold of 300 Da, the ash content can be reduced from 8 to 10% initially to values of 5 to 7%.

In order to measure the ash content, the skilled person will use any method that is well known in the field. Preferably, a person skilled in the art will proceed as follows:
Weigh a pea sample P1
Place the sample in an oven at 550° C. for 24 hours
Weigh the new sample weight P2

Ash content=(P2/P1)*100.

The method according to the invention can also comprise an optional step 7) of drying the pea or faba bean protein. Generally, this drying step is carried out so as to reach a solids content greater than 80%, preferentially greater than 90%, most preferentially greater than 94% by weight of solids relative to the weight of said pea protein. To this end, any technique well known to those skilled in the art can be used, for instance freeze-drying, flash drying or drying on a drying cylinder, or atomization. The process may also comprise a step of milling or micronizing. Atomization is the preferred technology, in particular multiple-effect atomization. The pea or faba bean protein may be in the form of a powder having a particle size d50, which can vary widely, for example from 10 μm to 500 μm, generally from 50 μm to 150 μm.

In the present invention, “d50” means the particle size measured in microns separating two populations by number, containing respectively 50% and 50% of the total amount of particles of the protein composition.

For performing this d50 measurement, a laser particle size analyzer is preferentially used, even more preferentially the Mastersizer 2000 from Malvern. The parameters used are as follows: Liquid application, dispersed in ethyl alcohol; Refractive index: 1.52; Absorption index: 0.1; No ultrasound used.

Preferably, drying step 7) can comprise a first sub-step of concentration by evaporation, followed by the actual drying step. This evaporation is conventionally carried out using any suitable technique well known to the skilled person, such as vacuum evaporation. The target dry matter content is between 15% and 30%, preferentially between 20% and 23%. This evaporation both eliminates undesirable volatile compounds and optimizes the drying step.

Steps 1) to 7) of the method can be carried out in this precise order but, depending on the needs of the skilled person, other optional steps can be implemented, such as pH adjustment.

### Pea or Faba Bean Protein

Another object of the invention relates to a composition of pea or faba bean protein that can be obtained by the method of the invention.

Another object of the invention relates to the composition of pea or faba bean protein characterized in that it comprises a protein content of between 50% and 70% expressed in grams of protein per 100 g of dry matter, said proteins consisting of a mixture of globulins and albumins, and between 3% and 15% of soluble fibers derived from pea galactooligosaccharides.

“Composition of pea protein” means a composition that comprises mainly, but not exclusively, pea proteins. Such a composition may contain residual impurities such as, for example, minerals, sugars, etc.

“Composition of faba bean protein” means a composition that comprises mainly, but not exclusively, faba bean proteins. Such a composition may contain residual impurities such as, for example, minerals, sugars, etc.

The term “pea” should be understood in the present application as all the wild varieties of “smooth pea” and all the mutant varieties of “smooth pea” and “wrinkled pea”. The term “pea” is considered here in its broadest accepted use and includes in particular all the varieties of “smooth pea” and “wrinkled pea” and all the mutant varieties of “smooth pea” and “wrinkled pea”, regardless of the uses for which said varieties are usually intended (human food, animal feed and/or other uses). The term “pea” in the present application includes pea varieties belonging to the Pisum genus and more particularly to the species sativum and aestivum. Said mutant varieties are in particular those named “r mutants”, “rb mutants”, “rug 3 mutants”, “rug 4 mutants”, “rug 5 mutants” and “lam mutants” as described in the article by C-L HEYDLEY et al., entitled “Developing novel pea starches” Proceedings of the Symposium of the Industrial Biochemistry and Biotechnology Group of the Biochemical Society, 1996, pages 77-87.

The term “protein” should be understood in the present application to mean the macromolecules formed from one or more polypeptide chains consisting of a sequence of amino acid residues bonded to one another by peptide bonds. In the particular context of pea proteins, the present invention relates more particularly to globulins (about 50-60% by weight of the pea proteins) and albumins (20-25% by weight of the pea proteins).

For the purposes of the present invention, “globulins” means proteins that are soluble in neutral saline solutions. Pea or faba bean globulins are mainly subdivided into three sub-families: legumins, vicilins and convicilins.

The term “albumin”, for the purposes of the present invention, means proteins that are soluble in pure water. Pea albumins, which are present in pea or faba bean proteins at about 20%, are mainly subdivided into two families named PA1 and PA2. Pea or faba bean albumins are mainly subdivided into two families named PA1 and PA2.

Generally, the protein content by weight of the pea or faba bean protein according to the invention is between 50% and 70% expressed in grams of protein per 100 g of dry matter. The protein content is the N6.25 content, calculated using the Dumas method. Preferably, the protein content is between 50% and 70%, comprising 50%, 51%, 52%, 53%, 54%, 55%, 56%, 57%, 58%, 59%, 60%, 61%, 62%, 63%, 64%, 65%, 66%, 67%, 68%, 69 or 70%.

The pea or faba bean protein according to the invention obviously generally comprises other minority constituents other than proteins, such as starch, lipids, fibers, and/or sugars.

Generally, the total starch content in the pea or faba bean protein produced according to the method of the invention is between 0% to 0.5%, for example from 0.1% to 0.4%, especially from 0.2 to 0.3%. This total starch content can be measured using the AOAC 996.11 method. The residual starch contents can therefore be 0%, 0.1%, 0.2%, 0.3%, 0.4%, 0.5%. This particularly low starch content is characteristic of the pea or faba bean protein according to the invention. Pea or faba bean protein concentrates obtained by turboseparation contain more starch, for example between 2 and 5% according to the Feedipedia web page (https://www.feedipedia.org/node/7439), but more often up to more than 10% as will be exemplified later in this application in commercial concentrates. The presence of this residual starch will have an impact not only on the nutritional character of the product, but also on its functional properties, such as for example greater viscosity when heated.

Generally, the total lipid content is from 0% to 15%, for example from 1% to 10%. The total lipid content can be determined by the AOAC 996.06 acid hydrolysis method.

Preferably, the dry weight ratio of globulins/albumins is between 70:30 and 90:10, preferentially between 75:25 and 85:15.

Preferably, the pea or faba bean protein according to the invention has a degree of hydrolysis, or DH, of between 6% and 8%, preferentially between 6.5% and 7.5%. The degree of hydrolysis values will thus be 6.0%; 6.1%; 6.2%; 6.3%; 6.4%; 6.5%; 6.6%; 6.7%; 6.8%; 6.9%; 7.0%; 7.1%; 7.2%; 7.3%; 7.4%; 7.5%; 7.6%; 7.7%; 7.8%; 7.9% or 8.0%.

The expression “degree of hydrolysis”, for the purposes of the present invention, means the percentage ratio between the amount of amine (or carboxylic) functions of the free amino acids and the total amount, including the free functions and those engaged in a peptide bond (chemical bond characteristic of proteins resulting from the association of a carboxylic function of a first amino acid and an amine function of a second). For a protein composition consisting of the chain of all its amino acids and thus having only one free amine function and one free carboxylic function, this degree of hydrolysis will be 0%. Conversely, for a protein composition in which the same amino acids are all “free”, that is their two amine and carboxylic functions are not involved in peptide bonds, this degree of hydrolysis will be 100%.

There are several methods for quantifying the degree of hydrolysis. They all consist mainly of the colorimetric assay of the free amine (or carboxylic) functions, followed by the performance of a hydrolysis aimed at destroying all the peptide bonds and finally of a colorimetric assay of the total amine (or carboxylic) functions. The calculated percentage of the free amines (or carboxyls) to the total amines gives the degree of hydrolysis. Any well-known method can be used such as the so-called TNBS method or the OPA method. In the present invention, the OPA method is preferred, a measurement procedure for which is described hereunder:

The content of amino nitrogen (free NH₂) is determined first of all on the sample of proteins according to the invention with the MEGAZYME kit (reference K-PANOPA). The protein nitrogen content (total nitrogen) of the sample is also determined. It is then possible to calculate the degree of hydrolysis.

### Determining the Content of Amino Nitrogen:

The “amino nitrogen” groups of the free amino acids in the sample react with the N-acetyl-L-cysteine and o-phthaldialdehyde (OPA) to form isoindole derivatives.

The amount of isoindole formed during this reaction is stoichiometric with the amount of free amino nitrogen. It is the isoindole derivative which is measured by the increase in absorbance at 340 nm.

A test specimen P*, exactly weighed, of the sample to be analyzed is introduced into a 100 mL beaker. This test specimen will be from 0.5 to 5.0 g based on the amino nitrogen content of the sample. Approximately 50 ml of distilled water is added, homogenization is carried out and the mixture is decanted into a 100-ml graduated flask. 5 ml of 20% sodium dodecyl sulfate (SDS) are added, and the mixture is supplemented with distilled water to reach a volume of 100 ml. Stirring is carried out for 15 minutes with a magnetic stirrer at 1000 rpm. A solution no. 1 is prepared by dissolving a tablet from bottle 1 of the Megazyme kit in 3 ml of distilled water and stirring is carried out until it is completely dissolved. It is necessary to provide one tablet per test. Solution no. 1 is prepared immediately before use.

A blank, a standard and a sample are prepared directly in the spectrophotometer cuvettes under the following conditions:
blank: add 3.00 mL of solution No. 1 and 50 μL of distilled water
standard: add 3.00 mL of solution No. 1 and 50 μL of bottle 3 of the Megazyme kit
sample: introduce 3.00 mL of solution No. 1 and 50 μL of sample preparation.

The content of each cuvette is mixed and the measure of absorbance (A1) of the solutions is taken after approximately 2 mn in the spectrophotometer at 340 nm (spectrophotometer equipped with cuvettes with 1.0 cm of optical path, able to measure at a wavelength of 340 nm, and verified according to the procedure described in the related manufacturer's technical manual).

The reactions are then immediately initiated by adding 100 μl of solution no. 2, which corresponds to the OPA solution of bottle 2 of the Megazyme kit in each spectrophotometer cuvette.

The content of each cuvette is mixed and they are then placed in darkness for approximately 20 minutes.

The measure of absorbance A2 of the blank, the standard and the sample are then taken from the spectrophotometer at 340 nm.

The free amino nitrogen content, expressed as percentage by weight relative to the weight of the product, is given by the following formula:

$$\% amino nitrogen = \frac{( Δ Aech - Δ Ablc ) \times 3.15 \times 14.01 \times V \times 100}{6803 \times 0.05 \times m \times 1000}$$

$$\% amino nitrogen = \frac{( Δ Aech - Δ Ablc ) \times 12.974 \times V}{m \times 1000}$$

where:
ΔAech=Aech2−Aech1
ΔAblc=Ablc2−Ablc1
Aech2=absorbance of sample after adding solution no. 2
Aech1=absorbance of sample after adding solution no. 1
Ablc2=absorbance of blank after adding solution no. 2
Ablc1=absorbance of blank after adding solution no. 1
V=volume of flask
m=mass of test sample in g
6803=extinction coefficient of isoindole derivative at 340 nm (in L·mol⁻¹·cm⁻¹).
14.01=molar mass of nitrogen (in g·mol⁻¹)
3.15=final volume in the cuvette (in mL)
0.05=test sample in the cuvette (in mL)

### Determining the Content of Protein Nitrogen:

The content of protein nitrogen is determined according to the DUMAS method according to standard ISO 16634-2016. It is expressed as percentage by weight relative to the weight of the product.

### Calculation of the Degree of Hydrolysis

The degree of hydrolysis (DH) is calculated with the following formula:

$$D H = \frac{\% amino nitrogen}{\% protein nitrogen} \times 100$$

The pea or faba bean protein according to the invention contains between 3% and 15% soluble fiber, preferentially between 4% and 8% derived from pea galactooligosaccharides. The content can therefore be between 3%, 4%, 5%, 6% 7%, 8%, 9%, 10%, 11%, 12%, 13%, 14% and 15%.

Preferably, the soluble fibers derived from galactooligosaccharides of the pea or faba bean are selected from the list containing melibiose, manninotriose, verbascotetraose and mixtures thereof.

The combination of protein, composed of a mixture of globulin and albumin, and defructosylated soluble fibers creates a unique ingredient of undeniable interest. With this single ingredient, it is possible to provide both a quality protein and an amount of soluble fibers. Furthermore, according to the thesis “PREVENTION AND TREATMENT OF IRON DEFICIENCY ANEMIA IN WOMEN AND CHILDREN: IRON HOMEOSTASIS AND OPTIMIZING ABSORPTION USING PREBIOTICS AND BREAST MILK COMPONENTS” (Giorgetti, 2022), iron absorption is increased when consumed with GOS.

The properties of the pea or faba bean protein can vary widely, depending on the parameters of the method previously described and as shown in the Examples section.

According to one embodiment, the dried pea or faba bean protein has a solubility at pH 7 ranging from 10 to 99%. The solubility may have all the intermediate amounts (that is, 11%, 12%, 13% . . . 97%, 98%, 99%) and the skilled person will know, on the basis of the method indications given hereinbefore and in the examples section, how to modify the parameters of the method within the indicated ranges in order to achieve the desired solubility. Advantageously, the dried pea or faba bean protein has a solubility at pH 7.0 and at pH 4.0 ranging from 40% to 55%.

### Solubility: Test A

As regards solubility, this is determined using the TEST A method described below:

### Measurement of Solubility in Water

This measurement is based on diluting the sample in distilled water, centrifuging it and analyzing the supernatant.

### Procedure:

Introduce 150 g of distilled water into a 400 mL beaker at 20° C.±2° C., mix with a magnetic stirrer bar and add precisely 5 g of the sample to be tested.
Adjust the pH to the desired value with 0.1 N NaOH or HCl (pH 7), or do not adjust it.
Make up to 200 g with water.
Mix for 30 minutes at 1000 rpm and centrifuge for 15 minutes at 3000 g.
Collect 25 g of supernatant.
Introduce into a previously dried and tared crystallizer.
Place in an oven at 103° C.±2° C. for 1 hour.
Then place in a desiccator (with desiccant) to cool to ambient temperature and weigh.

The soluble dry matter content, expressed in % by weight, is given by the following formula:

$$[ Math . 1 ]$$

$$( m 1 - m 2 ) \times ( 200 + P ) \times 100$$

$$… … … … … … … = \% solubility$$

$$P 1 \times P$$

Where:
P=weight, in g, of the sample=5 g
m1=weight, in g, of the dried crystallizer
m2=weight, in g, of the empty crystallizer
P1=weight, in g, of the collected sample=25 g

According to one embodiment, the dried pea or faba bean protein can have gelling power. This gelling power can range from 1 to 100 Pa, preferentially between 10 and 90 Pa, even more preferentially between 30 and 60 Pa.

### Gelling Power: Test B

The term “gelling power” refers to the functional property which consists of the capacity of a protein composition for forming a gel or a network, which increases the viscosity and generates a state of matter between the liquid and solid states. It is also possible to use the term “gel strength”. To quantify this gelling power, it is thus necessary to generate this network and to evaluate its strength. To carry out this quantification, in the present invention, test B is used, the description of which is as follows:
1) Solubilizing at 60° C.+/−2° C. the protein composition tested in water containing 15%+/−2% solids content at pH 7;
2) Stirring for 5 minutes at 60° C.+/−2° C.;
3) Cooling to 20° C.+/−2° C. and stirring for 24 hours at 350 rpm; 4) Placing the suspension in a controlled-stress rheometer equipped with a concentric cylinder;
5) Measuring elastic modulus G′ and viscous modulus G″ using the following temperature profile:
a. Phase 1: Measuring parameter G′1 after stabilizing at 20° C.+/−2° C. and heating at a temperature of 20° C.+/−2° C. to a temperature of 80° C.+/−2° C. in 10 minutes;
b. Phase 2: Stabilizing at a temperature of 80° C.+/−2° C. for 110 minutes;
c. Phase 3: Cooling from a temperature of 80° C.+/−2° C. to a temperature of 20° C.+/−2° C. in 30 min and measuring G′2 after stabilization at 20° C.+/−2° C.;
6) Calculating gelling power equal to G′2−G′1.

In a preferred manner, the controlled stress rheometers are chosen from the models DHR 2 (TA, Instruments) and MCR 301 (Anton Paar), with a spindle of concentric cylinder type. They are equipped with a temperature regulation system based on the Peltier effect. In order to avoid evaporation problems at high temperature, liquid paraffin is added on top of the samples.

For the purposes of the invention, a “rheometer” is a laboratory machine for taking measurements regarding the rheology of a fluid or a gel. It applies a force to the sample. Generally of characteristic small dimensions (very small mechanical inertia of the rotor), it allows fundamental study of the mechanical properties of a liquid, a gel, a suspension, a paste, etc., in response to an applied force.

The so-called “controlled stress” models make it possible, by the application of a sinusoidal stress (oscillation mode), to determine the intrinsic viscoelastic values of matter, which notably are dependent upon time (or angular velocity ω) and upon temperature. In particular, this type of rheometer makes it possible to access the complex modulus G*, which itself makes it possible to access the moduli G′ or elastic part and G″ or viscous part;

The first three steps consist in resuspending the protein in water, using precise conditions making it possible to maximize the subsequent measurement.

The water selected is preferentially reverse osmosis water, but it is also possible to use drinking water.

Its temperature is 60° C.±2° C. during the initial resuspension (1st and 2nd steps) and then 20° C.±2° C. after solubilization for 24 h and cooling before the measurement (3rd step). In general and unless indicated otherwise, when a temperature is given in the present description, it always comprises a variation of ±2° C., for example 20° C.±2° C. or 80° C.±2° C.

A defined amount of protein is added to said water so as to obtain a suspension containing 15%±2% of solids. To do this, equipment such as beakers and stirring bars, well known to those skilled in the art, are used. A volume of 50 mL is stirred for at least 10 h at 350 rpm at room temperature. In general and unless indicated otherwise, the solids contents given in the present description always comprise a variation of ±2%, for example 15%±2%. The pH is adjusted to 7±0.5 using a pH-meter and acid-base reagents, as is well known in the prior art.

The fourth step consists in introducing the sample into the rheometer, and covering said sample with a thin layer of oil in order to limit the evaporation.

During the fifth step, the following temperature protocol is then applied: a. Phase 1: heating from a temperature of 20° C.±2° C. to a temperature of 80° C.±2° C. in 10 minutes; b. Phase 2: stabilization at a temperature of 80° C.±2° C. for 110 minutes; c. Phase 3: cooling from a temperature of 80° C.±2° C. to a temperature of 20° C.±2° C. in 30 min.

The measurement of the parameter G′ is performed continuously during this protocol and is recorded.

The sixth and last step of test B consists in exploiting the recording.

Two values are extracted: G′1=value of G′ at the start of phase 1 after stabilization at 20° C.±2° C. and G′2=value of G′ at the end of phase 3 after stabilization at 20° C.±2° C.

The gelling power is equal to G′2−G′1.

According to one optional variant, the pea or faba bean protein is an enzymatically modified protein. With enzymatically modified protein, the skilled person refers to a protein whose protein structure has been deliberately modified by the addition to the protein of at least one enzyme capable of modifying the protein structure. This enzyme can be selected from proteases, peptidases and deamidation enzymes, for example those of the EC type 3.5.1 such as glutaminase or deamination enzymes, for example those of the EC type 3.5.3 such as peptidylarginine deiminase. These protein-modifying enzymes are known for modifying the physicochemical and/or organoleptic properties of the protein. For example, it is known from document WO2019/233920 A1 that peptidylarginine deiminase reduces the astringency, especially the astringency of rapeseed protein. If the method comprises proteolysis of the protein-enriched fraction, this can modify the degree of hydrolysis (DH) of the protein. Preferably, the degree of hydrolysis is less than 15%, advantageously less than 10%, preferably less than 6%, for example between 3% and 5%. A person skilled in the art will know how to adapt the enzymatic proteolysis conditions, or even will not carry out such a step in order to obtain the desired DH. According to a preferred variant of the invention, the pea or faba bean protein is not enzymatically modified by deamination. According to another preferred variant of the invention, the protein is not enzymatically modified. One advantage of the invention is that it is possible to modify the organoleptic properties of pea protein, and especially to give it a milky flavor profile, without even needing to modify the protein enzymatically. According to one embodiment, the invention also makes it possible to provide proteins with an unmodified primary structure.

### Use of Pea Protein

Another object of the invention is the use of the pea or faba bean protein of the invention for producing food or beverage products, especially plant-based alternatives to milk.

Generally, the pea or faba bean protein of the invention can be used in food products and beverages that may include it in an amount of up to 100% by weight with respect to the total dry weight of the food or beverage product, for example in an amount ranging from about 1% by weight to about 80% by weight with respect to the total dry weight of the food or beverage product. All intermediate amounts (that is, 2%, 3%, 4% . . . 77%, 78%, 79% by weight relative to the total weight of the food or beverage product) can be used, as well as all the intermediate ranges based on these quantities. These food and beverage products can be adapted to vegetarian or vegan populations.

A particularly interesting use of the protein of the invention relates to its use in beverages which have a more pleasant taste than those obtained from other commercially available pea proteins. The pea or faba bean protein of the invention can advantageously be used to produce beverages, in particular milk alternatives, or in other words milk substitutes. Moreover, because of the milky flavor note due to the ingredient, these beverages can also have a more milky flavor note than a beverage not comprising said protein, which is an undeniable advantage for the production of plant-based alternatives to milk. In addition to improving the flavor, the invention also makes it possible to obtain a more velvety mouthfeel than when other pea or faba bean proteins are used, which is advantageous for beverages, and especially for plant-based alternatives to milk as animal milks generally also have a velvety mouthfeel.

In beverages, the protein content of these products can vary widely, and can also be a high-protein drink. The protein content can range, for example, from 1% to 12% by dry weight, with respect to the total weight of the beverage, especially from 3% to 10% by dry weight, with respect to the total weight of the beverage. The beverages can be of any type, and include plant-based alternatives to milk or milk substitutes, including barista-type milks and coffee creamers. These may also include other acidic or non-acidic ready-to-drink beverages such as carbonated drinks (including, but not limited to, carbonated soft drinks), non-carbonated drinks (including, but not limited to, non-carbonated soft drinks such as flavored waters, fruit juices and sweetened or unsweetened tea or coffee-based drinks), alcoholic beverages such as beers or spirits, smoothies, and beverage concentrates (including, but not limited to, liquid concentrates and syrups, as well as non-liquid “concentrates” such as freeze-dried and/or powdered preparations or “powder mixes”). It should be noted that in beverages, flavorings or masking agents are generally used to reduce the pea or faba bean flavor note or bitter aftertaste of the protein or else to flavor the beverage. One of the advantages of the pea or faba bean protein of the invention is that its use in place of conventional pea or faba bean proteins makes it possible to reduce this amount of flavoring or masking agent, or even to totally eliminate these constituents from the beverage altogether, while retaining a very satisfactory taste for the beverage. The beverages can also comprise hydrocolloids; however, since pea or faba bean protein provides a more velvety texture, it is possible to reduce or even eliminate the hydrocolloid content while retaining a velvety mouthfeel.

The food products to which it may relate comprise bakery products such as bread products (including, but not limited to, sourdough and unleavened breads, sandwich loaves, yeast breads, and unleavened breads such as baking soda breads), breads containing all types of wheat flour, breads containing all types of flour other than wheat flour (such as potato, rice, barley, spelt, and rye flours), gluten-free breads; mixes for the preparation of said bakery products; sweet bakery products (including, but not limited to, rolls, cakes, pies, pastries, waffles, pancakes, muffins, pancakes, and cookies); mixes for the preparation of said sweet bakery products; pie fillings and other sweet fillings (including, but not limited to, fruit pie fillings and nut pie fillings such as pecan pie fillings, as well as fillings for cookies, cakes, pastries, confectionery products and others, such as cream fillings); and snack bars (including, but not limited to, energy, cereal, nut, and/or fruit bars).

They can also be jellied desserts such as dessert crèmes, custards and puddings. Another type of dessert can also be frozen desserts (including, but not limited to, frozen dairy desserts such as ice cream—including regular ice cream, soft ice cream and all other types of ice cream—and frozen non-dairy desserts such as non-dairy ice cream, sorbet, and others).

Other products conventionally prepared using animal milk can also comprise the pea or faba bean protein of the invention to form substitutes. These products may be acidified and/or fermented with ferments, such as lactic, vegan, or mesophilic ferments. These may include yoghurts (including, but not limited to, full-fat, low-fat, and fat-free yoghurts, which may be milk protein-free and lactose-free). The term “yoghurt” also includes fromage frais and petits-suisses. It may also include cheese substitutes such as cheese spreads, melted cheeses, pressed cooked and uncooked cheeses, soft cheeses, spun cheeses, blue cheeses; it may include Emmental, string cheese, ricotta, provolone, Parmesan, Munster, mozzarella, Monterey Jack, Manchego, blue, fontina, feta, Edam, double Gloucester, Camembert, Cheddar, Brie, Asiago and havarti. It can also include other products such as plant-based butters and even crème fraîche.

Other products that can include the pea or faba bean protein of the invention are also sauces such as vinaigrettes or mayonnaise or ketchup-based sauces or syrups.

Likewise, the pea or faba bean proteins of the invention can be incorporated into confectionery products (including, but not limited to, jelly candies, soft candies, hard candies, chocolates, caramels, and gums); sweetened and unsweetened breakfast cereals (including, but not limited to, extruded cereals, flaked cereals, and puffed cereals); and cereal coating compositions for the preparation of breakfast cereals. These may also include sweet spreads (including, but not limited to, jellies, jams, nut butters such as peanut butter, spreads, and other spreadable products).

The pea or faba bean proteins of the invention can also be used as flavor carriers or encapsulants.

Other types of food and beverages that are not mentioned herein but which conventionally comprise one or more proteins can also be envisaged in the context of the present invention. In particular, animal feed (such as pet food) is explicitly envisaged.

The pea or faba bean protein can also be used, optionally after texturing, in meat substitutes such as emulsified sausages or hamburgers, or else in fish or seafood substitutes. It can also be used in egg-replacement formulations or in the production of protein products such as tofu or tempeh. Textured proteins generally refers to proteins textured by extrusion, i.e. dry extrusion or textured vegetable protein, or high moisture extrusion. The extruders can be single-screw, twin-screw or multi-screw. In the case of twin-screw extrusion, the extrusion can be co-rotating or counter-rotating. As examples of multi-screw extrusion, mention may be made of the planetary extruder and the ring extruder. It is also possible to cite other more specific technologies such as shear cell technology, microextrusion or else 3D printing.

Food products or beverages can be used especially in specialized nutrition, for example for specific populations, such as babies or infants, children, adolescents, adults, the elderly, athletes, people suffering from a disease, etc. These may be meal-replacement formulas or complete nutritional beverages, for example for weight management or clinical nutrition (e.g. tube feeding or enteral nutrition).

The pea or faba bean protein can be used as a single source of proteins, but can also be used in combination with other additional plant or animal proteins. These additional proteins may be hydrolyzed or non-hydrolyzed. These additional proteins are generally in the form of concentrates or isolates. A distinction is made between concentrates and isolates according to their protein content: concentrates generally have protein contents between 50% and 70% and isolates have protein contents greater than 70%, preferentially between 80% and 90%, respectively. The term “plant protein” denotes all the proteins derived from cereals, oleaginous plants, leguminous plants and tuberous plants, as well as all the proteins derived from algae and microalgae or fungi, used alone or as a mixture, selected from the same family or from different families. “Leguminous plants” generally refers, in the present application, to the family of dicotyledonous plants of the Fabales order. Several leguminous plants are significant crop plants, such as soybean, beans, especially the mung bean, chickpea, faba bean, groundnut, cultivated lentil, cultivated alfalfa, various clovers, broad beans, locust bean, licorice, and lupin. The additional leguminous plant protein can be selected from these leguminous plants or else be a pea or faba bean protein such as that used in the invention. In the present application, the term “cereals” refers to plants cultivated from the family of grasses producing edible grains, for example wheat, oat, rye, barley, corn, sorghum, or rice. Tubers may be carrot, cassava, konjac, potato, Jerusalem artichoke, sweet potato. Oilseed plants are generally plants that produce seeds from which oil is extracted. Oilseed plants can be selected from sunflower, rapeseed, peanut, sesame, squash, or flax. The animal proteins can, for example, be egg or milk proteins, such as whey proteins, casein, or caseinate proteins. The pea or faba bean protein composition of the invention can thus be used in association with one or more of these proteins or amino acids in order to improve the nutritional properties of the end product, for example to improve the PDCAAS of the protein or to provide other functionalities.

Pea or faba bean protein can also be used for producing pharmaceutical products or else in fermentation, for example for producing fungal metabolites or cell culture metabolites.

The invention and its advantages will now be illustrated in the embodiments detailed in the examples section hereunder. It should be noted that these examples do not limit the present invention.

### EXAMPLES

### Example 1: Pea Protein Concentrate

About 1000 kg of peas were used. The external fibers of the peas were first separated from the seeds by crushing (mechanically separating the external hull and the pea seeds) and skinning (sorting the external hulls and the skinned seeds using compressed air). The seeds thus prepared were ground using an attrition mill in order to obtain a particle size such that 88% of the particles were less than 100 microns and 1.9% were greater than 315 microns. The resulting flour was mixed with water to produce a suspension of ground pea seed flour with a dry matter content of about 20%. This suspension of ground pea seeds is stirred for 5 minutes and fed into a decanter centrifuge (Flottweg Z3). The protein-enriched fraction was recovered from the overflow (about 7% dry matter content).

The protein-enriched fraction was adjusted to pH 6.6 using sodium hydroxide and hydrochloric acid, then heat-treated at 130° C. for 10 seconds and flash-cooled to about 60° C. The solution is then atomized on a Nubilosa atomizer (air inlet temperature=195° C.−air outlet temperature=95° C.). The pea protein powder named “Pea concentrate according to example 1” was then analyzed.

TABLE 1

| | | | | Pea protein |
|---|---|---|---|---|
| | | | | concentrate |
| | Pea | | | marketed by |
| | protein | NUTRALYS ® | | LA-VITA |
| | ac- | F85M | | under the |
| | cording | (Pea | VESTKORN | name “pea |
| | to ex- | protein | pea protein | protein |
| | ample 1 | isolate) | concentrate | concentrate” |
| Proteins | 62.0% | 85.10% | 54.0% | 50-55% |
| Carbohydrates | 24.0% | 4.2% | 30.6% | 12-15% |
| of which starch | 0.1% | 0.1% | 5.4% | 12-15% |
| of which | 1.6% | <0.1% | <0.1% | <0.1% |
| glucose | | | | |
| of which | 0.2% | <0.1% | <0.1% | <0.1% |
| fructose | | | | |
| of which | 7.8% | <0.1% | 2.7% | <0.1% |
| sucrose | | | | |
| of which | <0.1% | <0.1% | <0.1% | <0.1% |
| melibiose | | | | |
| of which | <0.5% | <0.1% | <0.1% | <0.1% |
| manninotriose | | | | |
| of which | 0.7% | <0.1% | 1.7% | <0.1% |
| raffinose | | | | |
| of which | 6.1% | <0.1% | 5.6% | <0.1% |
| stachyose | | | | |
| of which | 6.1% | <0.1% | 4.4% | <0.1% |
| verbascose | | | | |
| Lipids | 5.0% | 8.4% | 4.9% | ND |
| Ash | 9.0% | 4.3 | 5.5% | ND |
| Degree of | 7.4% | 4.5% | 4.9% | ND |
| hydrolysis DH | | | | |
| Solubility at | 48.8% | 60% | 15% | ND |
| pH 4 according | | | | |
| to Test A | | | | |
| Solubility at | 52.2% | 15% | 34% | ND |
| pH 7 according | | | | |
| to Test A | | | | |
| Gel strength | 47 | | | |
| according to | | | | |
| Test B | | | | |

ND means not detected. Where this refers to a measurement, ND means not measured.

The pea concentrate obtained according to the invention is particularly interesting because it contains around 60% protein on dry matter and has a solubility at pH 4 or 7 of over 45%.

It also contains 12.9% GOS which can be defructosylated according to the teaching in Example 2 to enrich it with this natural fiber.

The starch content of the pea concentrate obtained according to the invention (wet process) is lower than that of the tests of Pea protein concentrate marketed by LA-VITA under the name “pea protein concentrate” and the VESTKORN pea protein concentrate obtained by the dry process.

The combination of the content and the quality of proteins (globulins+albumins) and the presence of natural soluble fibers makes it a food ingredient of choice.

### Example 2: Defructosylation Reaction of Pea Proteins

We proceed as in Example 1 but a defructosylation step is performed on the protein fraction recovered from the overflow of the decanter centrifuge (Flottweg Z3) prior to the pH neutralization step.

The pH of the resulting protein-enriched fraction is rectified to 5.0 using hydrochloric acid and sodium hydroxide. The rectified protein-enriched fraction is heated and then temperature-regulated at 60° C. 0.2% enzyme on a dry weight basis of Sumizyme INV (invertase) is added. It is left to react for 40 min with stirring.

The rest of the method is identical to Example 1. The pH is then neutralized to 6.6. The solution is evaporated to 20% dry matter, undergoes HTST treatment at 130° C. for 10 seconds with a 60° C. flash-cool when exiting.

The solution is then atomized on a Nubilosa atomizer (air inlet temperature=195° C.−air outlet temperature=95° C.). The pea protein powder named “Pea concentrate defructosylated according to Example 2” was then analyzed.

TABLE 3

| | before | after |
|---|---|---|
| | defructosylation | defructosylation |
| Raffinose | 0.7% | <0.1 |
| Stachyose | 6.1% | <0.1 |
| Verbascose | 6.1% | 0.9% |
| Manninotriose | <0.5% | 2.5% |
| Melibiose | <0.1% | 0.9% |
| Fructose | 0.20% | 6.0% |

The GOS (in italics, about 12%) was hydrolyzed to defructosylated GOS (about 4.5%) and to fructose (6%). Fructose provides a sweet flavor that softens the bitterness of pea proteins.

It should be noted that the enzymatic treatment using invertase can be carried out further downstream, that is on the concentrate obtained in step 1.

1. A method for producing pea or faba bean protein comprising the following steps:
1. Preparing an aqueous suspension of ground pea or faba bean seeds in an aqueous solution, said preparation being optionally performed in the presence of a heat treatment;
2. Removing an insoluble fraction by solid/liquid separation of the aqueous suspension of ground pea or faba bean seeds obtained in step 1) to obtain a protein-enriched fraction; and
3. Defructosylating galactooligosaccharides from the protein-enriched fraction obtained in step 3) by enzymatic and/or fermentative means.

2. The method according to claim 1 wherein the aqueous suspension of pea or faba bean seeds of step 1 is obtained by adding a pea or faba bean flour obtained by dry milling prior to its dispersion in an aqueous solution.

3. The method according to claim 1 wherein when the pea or faba bean seeds are introduced as whole pea or faba bean seeds into the aqueous solution, step 1) of the method comprises a step of wet milling the aqueous composition formed between the pea or faba bean seeds and the aqueous solution in order to obtain the aqueous suspension of ground pea or faba bean seeds.

4. The method according to claim 1 wherein the aqueous suspension of ground pea or faba bean seeds of step 1) the optional heat treatment of step 1 comprises:
a) introducing pea or faba bean seeds or ground pea or faba bean seeds into an aqueous solution at a temperature of between 65° C. and 90° C. in order to obtain an aqueous composition comprising pea or faba bean seeds or ground pea or faba bean seeds; and
b) heat treating the aqueous composition obtained in step a) formed between the pea or faba bean seeds or the ground pea or faba bean seeds and the aqueous solution at a temperature of between 40° C. and 65° C. for 1 to 10 min.

5. The method according to claim 1 wherein the defructosylation of galactooligosaccharide (GOS) in step 3 is carried out with an enzyme selected from invertase, alpha-galactosidae, bet-fructosidase and any combinations thereof.

6. The method according to claim 1 wherein the defructosylation of galactooligosaccharide (GOS) is carried out with a microorganism of the genus Bacillus, preferentially Bacillus subtilis, preferentially a Bacillus subtilis strain as deposited with the CNCM on May 28, 2020 under number I-5515.

7. A composition of pea or faba bean protein comprising a protein content of between 50% and 70% expressed in grams of protein per 100 g of dry matter, said proteins consisting of a mixture of globulins and albumins, and between 3% and 15% of soluble fibers derived from pea or faba bean galactooligosaccharides.

8. The composition according to claim 7, wherein the globulin/albumin dry weight ratio is between 70:30 and 90:10, preferentially between 75:25 and 85:15.

9. The composition according to claim 7, wherein the soluble fibers derived from pea or faba bean galactooligosaccharides are selected from the list containing melibiose, manninotriose, verbascotetraose and mixtures thereof.

10. A use of said composition of pea or faba bean protein according to claim 7 or obtained according to a method for producing pea or faba bean protein comprising the following steps:
1. Preparing an aqueous suspension of ground pea or faba bean seeds in an aqueous solution, said preparation being optionally performed in the presence of a heat treatment;
2. Removing an insoluble fraction by solid/liquid separation of the aqueous suspension of ground pea or faba bean seeds obtained in step 1) to obtain a protein-enriched fraction; and
3. Defructosylating galactooligosaccharides from the protein-enriched fraction obtained in step 3) by enzymatic and/or fermentative means.
